(19) United States
(12) Patent Application Publication
(10) Pub. No.: US 2009/0280544 A1
(43) Pub. Date: Nov. 12, 2009

(54) METHOD FOR PRODUCING OPTICALLY ACTIVE 2-BENZYLOXYCYCLOHEXYLAMINE

(75) Inventors: Klaus Ditrich, Gonnheim (DE); Ute Reuther, Bissersheim (DE); Michael Bartsch, Neustadt (DE)

Correspondence Address: CONNOLLY BOVE LODGE & HUTZ, LLP, P O BOX 2207, WILMINGTON, DE, 19899, US

(73) Assignee: BASF SE, Ludwigshafen (DE)

(21) Appl. No.: 12/440,811

(22) PCT Filed: Sep. 12, 2007
(86) PCT No.: PCT/EP07/59594

§ 371 Date: Mar. 11, 2009

(30) Foreign Application Priority Data
Sep. 13, 2006 (EP) 06120597.7

(51) Int. Cl. C12P 13/00

(52) U.S. Cl. 435/128

(57) ABSTRACT

The present invention relates to a method for the enantioselective acylation of trans-2-benzyloxycyclohexylamine or cis-2-benzyloxycyclohexylamine, according to which an enantiomer mixture of 2-benzyloxycyclohexylamine is reacted with an acylation agent in the presence of a hydrolase. The invention also relates to a method for producing optically active trans-stereoisomers of 2-benzyloxycyclohexylamine.

The present invention relates to a process for the enantioselective acylation of trans- or cis-2-benzyloxycyclohexylamine and to a process for preparing optically active trans stereoisomers of 2-benzyloxycyclohexylamine.

Optically active compounds are of very great importance especially in the pharmaceutical industry sector because frequently only a particular optically active isomer has therapeutic activity. There is thus a continually growing demand for optically active compounds as precursors for the enantioselective synthesis of active ingredients. One of these key building blocks for synthesizing novel active ingredients is optically active trans-2-benzyloxycyclohexylamine.

WO 96/23894 describes in general a process for resolving the racemates of primary and secondary heteroatom-substituted amines by reaction with an ester in the presence of a hydrolase and subsequent separation of the enantioselectively acylated enantiomer of the heteroatom-substituted amine from the unreacted enantiomer. The enantiomeric purities of the products are, however, still unsatisfactory.

It was an object of the present invention to provide an efficient process for preparing substantially enantiomerically pure trans and also cis stereoisomers of 2-benzyloxycyclohexylamine. The process was intended in particular to allow the preparation of substantially enantiomerically pure (R,R)-2-benzyloxycyclohexylamine, especially in an enantiomeric purity of at least 99% ee, preferably at least 99.5% ee and in particular at least 99.9% ee.

It has surprisingly been found that optically active 2-benzyloxycyclohexylamine is obtained by enantioselective N-acylation of a mixture of enantiomers, in particular of the racemate of these amines in the presence of a hydrolase.

It has further been found that the respective cis or trans enantiomers are obtained in highly pure form when they are precipitated in the form of their acid addition salts by adding an acid (e.g. carboxylic acid, mineral acid).

The invention thus relates to a process (process A) for the enantioselective acylation of trans- or cis-2-benzyloxycyclohexylamine (I) in which a mixture of enantiomers of the trans stereoisomer or of the cis stereoisomer of the compound of the formula (I)

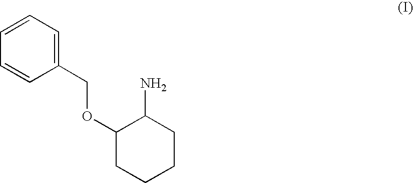

is reacted with an acylating agent in the presence of a hydrolase, resulting in a mixture in which one enantiomer of the trans or of the cis stereoisomer is present substantially in the acylated form, and the other enantiomer of the trans or of the cis stereoisomer is present substantially in the nonacylated form.

The invention further relates to a process (process B) for preparing optically active compounds of the formulae ((R,R)-I) and/or ((S,S)-I)

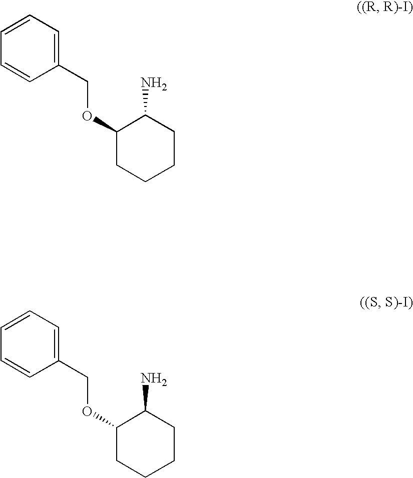

comprising the following steps:

(a) reacting a mixture of enantiomers of the trans stereoisomer of the compound of the formula (I) as defined above (i.e. mixture of the compounds ((R,R)-I) and ((S,S)-I)) with an acylating agent in the presence of a hydrolase, resulting in a mixture in which one enantiomer of the trans stereoisomer of the compound (I) is present substantially in the acylated form, and the other enantiomer of the trans stereoisomer of the compound (I) is present substantially in the nonacylated form;

(b) removing the nonacylated enantiomer of the trans stereoisomer of the compound (I) from the mixture obtained in step (a);

(c) hydrolyzing the substantially acylated enantiomer of the trans stereoisomer of the compound (I), obtained in step (b), to the corresponding nonacylated enantiomer.

The preferred features and embodiments of the process of the invention which are described below apply in each case independently or, in particular, in combination with one another.

The descriptors “trans” and “cis” describe in the context of the present invention the relative disposition of the two substituents on the cyclohexyl ring in relation to one another. The expression trans stereoisomer thus comprises (R,R)-2-benzyloxycyclo-hexylamine and (S,S)-2-benzyloxycyclohexylamine. The expression cis stereoisomer comprises the other diastereomer consisting of (R,S)-2-benzyloxycyclohexylamine and (S,R)-2-benzyloxycyclohexylamine.

The expression “enantioselective” describes a reaction in which the possible reaction products which are mirror images of each other are formed to unequal extents. In the context of the present invention, this expression describes reactions in which the desired enantiomer is formed preferably with at least 95% ee, particularly preferably with at least 96% ee, more preferably with at least 98% ee, even more preferably with at least 99% ee, in particular with at least 99.5% ee and specifically with at least 99.9% ee.

The expression “substantially enantiomerically pure” thus describes in the context of the present invention an enantiomeric purity of in each case at least 95% ee, preferably at least 96% ee, more preferably at least 98% ee, even more preferably at least 99% ee, in particular at least 99.5% ee and specifically at least 99.9% ee.

The unit “% ee” relates to the enantiomeric excess and is thus a measure of the enantiomeric purity, which is also referred to as optical purity. This is calculated from the difference of the molar proportions of the two enantiomers in a mixture of enantiomers. For example, 95% ee means that the proportion of the major enantiomer in the mixture of enantiomers is 97.5%, and the proportion of the compound which is the mirror image thereof is 2.5%.

The enantioselective acylation of the trans stereoisomer of the compound (I) preferably results in a mixture in which the (S,S) enantiomer is present substantially in nonacylated form, and the (R,R) enantiomer is present substantially in acylated form.

The expression “substantially nonacylated enantiomer” describes the situation where at least 95%, preferably at least 97%, in particular at least 98% of this enantiomer is nonacylated. Analogously, the expression “substantially acylated enantiomer” means that at least 95%, preferably at least 97%, particularly preferably at least 98%, of this enantiomer is in acylated form.

The expression “substantially nonacylated (S,S) enantiomer of the compound (I)” is correspondingly to be understood to mean that at least 95%, preferably at least 97%, in particular at least 98% of the (S,S) enantiomer of the compound (I) is nonacylated. Analogously, the expression “substantially acylated (R,R) enantiomer of the compound (I)” means that at least 95%, preferably at least 97%, particularly preferably at least 98%, of the (R,R) enantiomer of the compound (I) is in acylated form.

The hydrolase employed in the processes of the invention is preferably a protease and in particular a lipase. This brings about a selective N-acylation (amidation) of only one of the respective two enantiomers of the trans stereoisomer or of the cis stereoisomer of the compound (I), preferably of the trans stereoisomer of the compound (I). It particularly preferably brings about the selective amidation of the (R,R) enantiomer of the compound (I).

The hydrolase is preferably obtained from a microorganism, particularly preferably from a bacterium or a yeast. Hydrolases obtainable by recombinant processes are likewise suitable. The hydrolase can be used in purified or partially purified form or in the form of the microorganism itself. Processes for obtaining and purifying hydrolases from microorganisms are well known to the skilled worker, e.g. from EP-A-1149849 or EP-A-1069183. The hydrolase is preferably employed in purified form.

The hydrolase may be employed free (i.e. in native form) or immobilized. An immobilized enzyme means an enzyme which is fixed to an inert carrier. Suitable carrier materials and the enzymes immobilized thereon are disclosed in EP-A-1149849, EP-A-1 069 183 and DE-A 10019377 and from the references cited therein. The disclosure in these publications is incorporated herein by reference. Suitable carrier materials include for example clays, clay minerals such as kaolinite, diatomaceous earth, perlite, silicon dioxide, aluminum oxide, sodium carbonate, calcium carbonate, cellulose powder, anion exchanger materials, synthetic polymers such as polystyrene, acrylic resins, phenol-formaldehyde resins, polyurethanes and polyolefins such as polyethylene and polypropylene. The carrier materials are normally employed in a finely divided particulate form to prepare the carrier-bound enzymes, with preference for porous forms. The particle size of the carrier material is normally not more than 5 mm, in particular not more than 2 mm (grading curve).

Lipases (triacylglycerol acylhydrolases; EC 3.1.1.3) are preferably employed. Of these, lipases obtained from bacteria of the genera Burkholderia or Pseudomonas or from yeasts of the genus Candida are preferred.

Examples of Burkholderia species are Burkholderia ambifaria (e.g. strains ATCC BAA-244, CCUG 44356, LMG 19182), Burkholderia andropogonis (e.g. strains ATCC 23061, CCUG 32772, CFBP 2421, CIP 105771, DSM 9511, ICMP 2807, JCM 10487, LMG 2129, NCPPB 934, NRRL B-14296); Burkholderia caledonica (e.g. strains W50D, CCUG 42236, CIP 107098, LMG 19076); Burkholderia caribensis (e.g. strains MWAP 64, CCUG 42847, CIP 106784, DSM 13236, LMG 18531); Burkholderia caryophylli (e.g. strains ATCC 25418, CCUG 20834, CFBP 2429, CFBP 3818, CIP 105770, DSM 50341, HAMBI 2159, ICMP 512, JCM 9310, JCM 10488, LMG 2155, NCPPB 2151); Burkholderia cepacia (e.g. strains Ballard 717, 717-ICPB 25, ATCC 25416, CCUG 12691, CCUG 13226, CFBP 2227, CIP 80.24, DSM 7288, HAMBI 1976, ICMP 5796, IFO 14074, JCM 5964, LMG 1222, NCCB 76047, NCPPB 2993, NCTC 10743, NRRL B-14810); Burkholderia cocovenenans (e.g. strains ATCC 33664, CFBP 4790, DSM 11318, JCM 10561, LMG 11626, NCIMB 9450); Burkholderia fungorum (e.g. strains Croize P763-2, CCUG 31961, CIP 107096, LMG 16225); Burkholderia gladioli (e.g. strains ATCC 10248, CCUG 1782, CFBP 2427, CIP 105410, DSM 4285, HAMBI 2157, ICMP 3950, IFO 13700, JCM 9311, LMG 2216, NCCB 38018, NCPPB 1891, NCTC 12378, NRRL B-793); Burkholderia glathei (e.g. strains ATCC 29195, CFBP 4791, CIP 105421, DSM 50014, JCM 10563, LMG 14190); Burkholderia glumae (e.g. strains ATCC 33617, CCUG 20835, CFBP 4900, CFBP 2430, CIP 106418, DSM 9512, ICMP 3655, LMG 2196, NCPPB 2981, NIAES 1169); Burkholderia graminis (e.g. strains C4D1M, ATCC 700544, CCUG 42231, CIP 106649, LMG 18924); Burkholderia kururiensis (e.g. strains KP 23, ATCC 700977, CIP 106643, DSM 13646, JCM 10599, LMG 19447); Burkholderia mallei (e.g. strains ATCC 23344, NCTC 12938); Burkholderia multivorans (e.g. strains ATCC BAA-247, CCUG 34080, CIP 105495, DSM 13243, LMG 13010, NCTC 13007); Burkholderia norimbergensis (e.g. strains R2, ATCC BAA-65, CCUG 39188, CFBP 4792, DSM 11628, CIP 105463, JCM 10565, LMG 18379); Burkholderia phenazinium (e.g. strains ATCC 33666, CCUG 20836, CFBP 4793, CIP 106502, DSM 10684, JCM 10564, LMG 2247, NCIB 11027); Burkholderia pikettii (e.g. strains ATCC 2751 1, CCUG 3318, CFBP 2459, CIP 73.23, DSM 6297, HAMBI 2158, JCM 5969, LMG 5942, NCTC 11149); Burkholderia plantarii (e.g. strains AZ 8201, ATCC 43733, CCUG 23368, CFBP 3573, CFBP 3997, CIP 105769, DSM 9509, ICMP 9424 JCM 5492, LMG 9035, NCPPB 3590, NIAES 1723); Burkholderia pseudomallei (e.g. strains WRAIR 286, ATCC 23343, NCTC 12939); Burkholderia pyrrocinia (e.g. strains ATCC 15958, CFBP 4794, CIP 105874, DSM 10685, LMG 14191); Burkholderia sacchari (e.g. strains CCT 6771, CIP 107211, IPT 101, LMG 19450); Burkholderia solanacearum (e.g. strains A. Kelman 60-1, ATCC 11696, CCUG 14272, CFBP 2047, CIP 104762, DSM 9544, ICMP 5712, JCM 10489, LMG 2299, NCAIM B.01459, NCPPB 325, NRRL B-3212); Burkholderia stabilis (e.g. strains ATCC BAA-67, CCUG 34168, CIP 106845, LMG 14294, NCTC 13011); Burkholderia thailandensis (e.g. strains E 264, ATCC 700388, CIP 106301, DSM 13276); Burkholderia ubonensis (e.g. strains EY 3383, CIP 107078, NCTC 13147); Burkholderia vandii (e.g. strains VA-1316, ATCC 51545, CFBP 4795, DSM 9510, JCM 7957, LMG 16020); Burkholderia vietnamiensis (e.g. strains TVV 75, ATCC BAA-248, CCUG 34169, CFBP 4796, CIP 105875, DSM 11319, JCM 10562, LMG 10929).

Examples of Pseudomonas species are Pseudomonas aeruginosa (e.g. strains ATCC 10145, DSM 50071), Pseudomonas agarici (e.g. strains ATCC 25941, DSM 11810), Pseudomonas alcaligenes (e.g. strains ATCC 14909, DSM 50342), Pseudomonas amygdali (e.g. strains ATCC 337614, DSM 7298), Pseudomonas anguiliseptica (e.g. strains ATCC 33660, DSM 12111), Pseudomonas antimicrobica (e.g. strains DSM 8361, NCIB 9898, LMG 18920), Pseudomonas aspleni (e.g. strains ATCC 23835, CCUG 32773), Pseudomonas aurantiaca (e.g. strains ATCC 33663, CIP 106710), Pseudomonas aureofaciens (e.g. strains ATCC 13985, CFBP 2133), Pseudomonas avellanae (e.g. strains DSM 11809, NCPPB 3487), Pseudomonas azotoformans (e.g. strains CIP 106744, JCM 7733), Pseudomonas balearica (e.g. strains DSM 6083, CIP 105297), Pseudomonas beijerinsckii (e.g. strains ATCC 19372, DSM 6083), Pseudomonas beteli (e.g. strains ATCC 19861, CFBP 4337), Pseudomonas boreopolis (e.g. strains ATCC 33662, CIP 106717), Pseudomonas carboxyhydrogena (e.g. strains ATCC 29978, DSM 1083), Pseudomonas caricapapayae (e.g. strains ATCC 33615, CCUG 32775), Pseudomonas cichorii (e.g. strains ATCC 10857, DSM 50259), Pseudomonas cissicola (e.g. strains ATCC 33616, GCUG 18839), Pseudomonas citronellolis (e.g. strains ATGC 13674, DSM 50332), Pseudomonas coronafaciens (e.g. strains DSM 50261, DSM 50262), Pseudomonas corrugata (e.g. strains ATCG 29736, DSM 7228), Pseudomonas doudoroffii (e.g. strains ATCC 27123, DSM 7028), Pseudomonas echinoides (e.g. strains ATCC 14820, DSM 1805), Pseudomonas elongata (e.g. strains ATCC 10144, DSM 681 0), Pseudomonas ficuserectae (e.g. strains ATCC 35104, CCUG 32779), Pseudomonas flavescens (e.g. strains ATCC 51555, DSM 12071), Pseudomonas flectens (e.g. strains ATCC 12775, CFBB 3281), Pseudomonas fluorescens (e.g. strains ATCC 13525, DSM 50090), Pseudomonas fragi (e.g. strains ATCC 4973, DSM 3456), Pseudomonas fulva (e.g. strains ATCC 31418, CIP 106765), Pseudomonas fuscovaginae (e.g. strains CCUG 32780, DSM 7231), Pseudomonas gelidicola (e.g. strains CIP 106748), Pseudomonas geniculata (e.g. strains ATCC 19374, LMG 2195), Pseudomonas glathei (e.g. strains ATCC 29195, DSM 50014), Pseudomonas halophila (e.g. strains ATCC 49241, DSM 3050), Pseudomonas hibiscicola (e.g. strains ATCC 19867, LMG 980), Pseudomonas huttiensis (e.g. strains ATCC 14670, DSM 10281), Pseudomonas iners (e.g. strain CIP 106746), Pseudomonas lancelota (e.g. strains ATCC 14669, CFBP 5587), Pseudomonas lemoignei (e.g. strains ATCC 17989, DSM 7445), Pseudomonas lundensis (e.g. strains ATCC 19968, DSM 6252), Pseudomonas luteola (e.g. strains ATCC 43273, DSM 6975), Pseudomonas marginalis (e.g. strains ATCC 10844, DSM 13124), Pseudomonas meliae (e.g. strains ATCC 33050, DSM 6759), Pseudomonas mendocina (e.g. strains ATCC 25411, DSM 50017), Pseudomonas mucidolens (e.g. strains ATCC 4685, CCUG 1424), Pseudomonas monteilli (e.g. strains ATCC 700476, DSM 14164), Pseudomonas nautica (e.g. strains ATCC 27132, DSM 50418), Pseudomonas nitroreducens (e.g. strains ATCC 33634, DSM 14399), Pseudomonas oleovorans (e.g. strains ATCC 8062, DSM 1045), Pseudomonas oryzihabitans (e.g. strains ATCC 43272, DSM 6835), Pseudomonas pertucinogena (e.g. strains ATCC 190, CCUG 7832), Pseudomonas phenazinium (e.g. strains ATCC 33666, DSM 10684), Pseudomonas pictorum (e.g. strains ATCC 23328, LMG 981), Pseudomonas pseudoalcaligenes (e.g. strains ATCC 17440, DSM 50188), Pseudomonas putida (e.g. strains ATCC 12633, DSM 291), Pseudomonas pyrrocinia (e.g. strains ATCC 15958, DSM 10685), Pseudomonas resinovorans (e.g. strains ATCG 14235, CCUG 2473), Pseudomonas rhodesiae (e.g. strains CCUG 38732, DSM 14020), Pseudomonas saccharophila (e.g. strains ATCC 15946, DSM 654), Pseudomonas savastanoi (e.g. strains ATCC 13522, CFBP 1670), Pseudomonas spinosa (e.g. strains ATCC 14606), Pseudomonas stanieri (e.g. strains ATCC 27130, DSM 7027), Pseudomonas straminae (e.g. strains ATCC 33636, CIP 106745), Pseudomonas stutzeri (e.g. strains ATCC 17588, DSM 5190), Pseudomonas synxantha (e.g. strains ATCC 9890, CFBP 5591), Pseudomonas syringae (e.g. strains ATCC 19310, DSM 6693), Pseudomonas syzygii (e.g. strains ATCC 49543, DSM 7385), Pseudomonas taetrolens (e.g. strains ATCC 4683, CFBP 5592), Pseudomonas tolaasii (e.g. strains ATCC 33618, CCUG 32782), Pseudomonas veronii (e.g. strains ATCG 700272, DSM 11331), Pseudomonas viridiflava (e.g. strains ATCC 13223, DSM 11124), Pseudomonas vulgaris, Pseudomonas wisconsinensis and Pseudomonas spec. DSM 8246. Of these, lipases from Burkholderia glumae, Burkholderia plantarii, Burkholderia cepacia, Pseudomonas aeruginosa, Pseudomonas fluorescens, Pseudomonas fragi, Pseudomonas luteola, Pseudomonas vulgaris, Pseudomonas wisconsinensis and Pseudomonas spec. DSM 8246 are preferred. Lipases from Pseudomonas spec. DSM 8246 are particularly preferred.

Examples of Candida species are Candida albomarginata (e.g. strain DSM 70015), Candida antarctica (e.g. strain DSM 70725), Candida bacarum (e.g. strain DSM 70854), Candida bogoriensis (e.g. strain DSM 70872), Candida boidinii (e.g. strains DSM 70026, 70024, 70033, 70034), Candida bovina (e.g. strain DSM 70156), Candida brumptii (e.g. strain DSM 70040), Candida cacaoi (e.g. strain DSM 2226), Candida cariosilignicola (e.g. strain DSM 2148), Candida chalmersii (e.g. strain DSM 70126), Candida ciferii (e.g. strain DSM 70749), Candida cylindracea (e.g. strain DSM 2031), Candida ernobii (e.g. strain DSM 70858), Candida famata (e.g. strain DSM 70590), Candida freyschussii (e.g. strain DSM 70047), Candida friederichii (e.g. strain DSM 70050), Candida glabrata (e.g. strains DSM 6425,11226, 70614, 70615), Candida guillermondi (e.g. strains DSM 11947, 70051, 70052), Candida haemulonii (e.g. strain DSM 70624), Candida inconspicua (e.g. strain DSM 70631), Candida ingens (e.g. strains DSM 70068, 70069), Candida intermedia (e.g. strain DSM 70753), Candida kefyr (e.g. strains DSM 70073, 70106), Candida krusei (e.g. strains DSM 6128, 11956, 70075, 70079, 70086), Candida lactiscondensi (e.g. strain DSM 70635), Candida lambica (e.g. strains DSM 70090, 70095), Candida lipolytica (e.g. strains DSM 1345, 3286, 8218, 70561 or 70562), Candida lusitaniae (e.g. strain DSM 70102), Candida macedoniensis (e.g. strain DSM 70106), Candida magnoliae (e.g. strains DSM 70638, 70639), Candida membranaefaciens (e.g. strain DSM 70109), Candida multigemnis (e.g. strain DSM 70862), Candida mycoderma (e.g. strain DSM 70184), Candida nemodendra (e.g. strain DSM 70647), Candida nitratophila (e.g. strain USM 70649), Candida norvegica (e.g. strain DSM 70862), Candida parapsilosis (e.g. strains DSM 5784, 4237, 11224, 70125, 70126), Candida pelliculosa (e.g. strain DSM 70130), Candida pini (e.g. strain DSM 70653), Candida pulcherrima (e.g. strain DSM 70336), Candida punicea (e.g. strain DSM 4657), Candida pustula (e.g. strain DSM 70865), Candida rugosa (e.g. strain DSM 70761), Candida sake (e.g. strain DSM 70763), Candida silvicola (e.g. strain DSM 70764), Candida solani (e.g. strain DSM 3315), Candida sp. (e.g. strain DSM 1247), Candida spandovensis (e.g. strain DSM 70866), Candida succiphila (e.g. strain DSM 2149), Candida utilis (e.g. strains DSM 2361, 70163 or 70167), Candida valida (e.g. strains DSM 70169, 70178, 70179), Candida versatilis (e.g. strain DSM 6956), Candida vini (e.g. strain DSM 70184) and Candida zeylanoides (e.g. strain DSM 70185).

Lipases from yeasts of the genus Candida, in particular from Candida antarctica, are particularly preferably employed in the process of the invention. Accordingly, in a specific embodiment of the process of the invention, Lipase B from Candida antarctica is used. Preference is given to the immobilized form of this lipase, e.g. Lipase B from Candida antarctica immobilized on acrylic resin, which is commercially obtainable for example under the name “Novozyme 435®”.

The amount of hydrolase to be employed in the enantioselective acylation in the process of the invention depends on the nature thereof and the activity of the enzyme preparation. The amount of enzyme optimal for the reaction can easily be ascertained by simple preliminary tests. Ordinarily, about 1000 units of hydrolase are employed per mmol of mixture of enantiomers to be separated.

The unit “units” describes the activity of the hydrolase and refers to the amount of a reference compound which is converted by the hydrolase under defined standard conditions.

The acylating agents employed in the process of the invention are preferably selected from those in which the acid component has an electron-rich heteroatom which is for example selected from fluorine, nitrogen, oxygen and sulfur atoms, in the vicinity of the carbonyl carbon atom. The acylating agent is preferably an ester. The acylating agent is particularly preferably selected from esters whose acid component has an oxygen-, nitrogen-, fluorine- or sulfur-containing group in the α, β or γ position relative to the carbonyl carbon atom. Particularly preferred acylating agents are those in which the heteroatom itself is bonded in the α, β or γ position and in particular in the α position relative to the carbonyl carbon atom.

The oxygen-containing group is for example a hydroxy group or an alkoxy group. The nitrogen-containing group is for example an amino group, while the sulfur-containing group may be the thiol group (SH) or alkylthio groups.

The alcohol component of the ester is preferably derived from linear or branched C₁-C₁₀ alcohols which may be substituted or, preferably, unsubstituted. However, the alcohol component is particularly preferably derived from secondary alcohols such as isopropanol, 2-butanol, 2- or 3-pentanol and the like. It is specifically derived from isopropanol.

Particularly suitable esters are those of the formula (III)

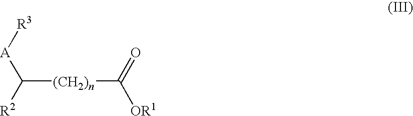

in which
R¹ is C₁-C₁₀-alkyl,
R² is hydrogen or C₁-C₁₀-alkyl,
R³ is hydrogen, C₁-C₁₀-alkyl or phenyl which is optionally substituted by NH₂, OH, C₁-C₄-alkoxy or halogen,
A is O, S or NR⁴, preferably O,
R⁴ is hydrogen, C₁-C₁₀-alkyl or phenyl which is optionally substituted by NH₂, OH, C₁-C₄-alkoxy or halogen, and
n is 0, 1 or 2.

R¹ is preferably linear or branched C₁-C₄-alkyl. R¹ is derived in particular from secondary alcohols and is accordingly particularly preferably a C₃-C₄-alkyl group which is bonded via a tertiary carbon atom, such as isopropyl or 2-butyl. R¹ is specifically isopropyl.

R² is preferably hydrogen or C₁-C₄-alkyl and in particular hydrogen.

R³ is preferably C₁-C₄-alkyl, particularly preferably methyl or ethyl and specifically methyl.

A is preferably O.

The expression “Cl₁-C₄-alkyl” describes in the context of the present invention a linear or branched alkyl radical having 1 to 4 carbon atoms, such as methyl, ethyl, propyl, isopropyl, n-butyl, sec-butyl, isobutyl or tert-butyl.

The expression “C₁-C₁₀-alkyl” is a linear or branched alkyl radical having 1 to 10 carbon atoms. Examples thereof are, besides the aforementioned C₁-C₄-alkyl radicals, pentyl, neopentyl, hexyl, heptyl, octyl, 2-ethylhexyl, nonyl, neononyl, decyl and neodecyl.

The expression “C₁-C₄-alkoxy” describes an alkyl radical linked via oxygen and having 1 to 4 carbon atoms. Examples thereof are methoxy, ethoxy, propoxy, isopropoxy, n-butoxy, sec-butoxy, isobutoxy and tert-butoxy.

The expression “C₁-C₁₀ alcohol” represents an aliphatic hydrocarbon having 1 to 10 carbon atoms which is substituted by at least one hydroxy group. C₁-C₁₀-alcohol preferably represents an alkane substituted by a hydroxy radical. Examples thereof are methanol, ethanol, propanol, isopropanol, butanol, sec-butanol, isobutanol, tert-butanol, pentanol, hexanol, heptanol, octanol, 2-ethylhexanol, nonanol and decanol.

In the context of the present invention, halogen is preferably fluorine, chlorine or bromine, particularly preferably fluorine or chlorine.

It is preferred to employ in the enantioselective acylation in the process of the invention from 1 to 3 mole equivalents, particularly preferably 1 to 2 mole equivalents, more preferably 1 to 1.5 mole equivalents and in particular 1 to 1.2 mole equivalents of the acylating agent based on the amount of enantiomer of the compound of the formula (I) which is acylated. The expression “mole equivalents” refers here to the number of carboxyl groups of the acylating agent in mole which are able to react with one mole of the enantiomer of the compound (I) which is acylated. Accordingly, when an ester of the formula (III) is used, preferably from 1 to 3 mol, particularly preferably 1 to 2 mol, more preferably 1 to 1.5 mol and in particular 1 to 1.2 mol of ester are employed per mol of the enantiomer of the compound of the formula (I) which is acylated. Alternatively, when the ester of the formula (III) is used, preferably from 0.5 to 1.5 mol, particularly preferably 0.5 to 1 mol, more preferably 0.5 to 0.75 and in particular 0.5 to 0.6 mol of ester is employed, based on 1 mol of the mixture of enantiomers to be separated, especially when this mixture is the racemate.

In a preferred embodiment of the process of the invention, the reaction in process A and in step (a) of process B takes place without dilution, i.e. without addition of aqueous or organic solvent.

In an alternatively preferred embodiment, the acylation in the process of the invention is carried out in a nonaqueous reaction medium. Nonaqueous reaction media mean reaction media which comprise less than 1% by weight, preferably less than 0.5% by weight of water, particularly preferably less than 0.1% by weight of water and in particular less than 0.05% by weight of water based on the total weight of the reaction medium. In this embodiment, the acylation is preferably carried out in an organic solvent. Examples of suitable solvents are aliphatic and alicyclic hydrocarbons, preferably having 5 to 8 carbon atoms, such as pentane, cyclopentane, hexane, cyclohexane, heptane, octane or cyclooctane, halogenated aliphatic hydrocarbons, preferably having 1 or 2 carbon atoms, such as dichloromethane, chloroform, tetrachloromethane, dichloroethane or tetrachloroethane, aromatic hydrocarbons such as benzene, toluene, the xylenes, chlorobenzene or dichlorobenzene, aliphatic acyclic and cyclic ethers, preferably having 4 to 8 carbon atoms, such as diethyl ether, methyl tert-butyl ether, ethyl tert-butyl ether, dipropyl ether, diisopropyl ether, dibutyl ether, tetrahydrofuran or dioxane, or mixtures of the aforementioned solvents. The aforementioned ethers and aromatic hydrocarbons are particularly preferably employed. Toluene is used in particular.

Reaction of the compounds of the formula (I) with the acylating agent ordinarily takes place at a reaction temperature below the deactivation temperature of the hydrolase employed, and preferably at −10° C. or above. It is particularly preferably in the range from 0 to 80° C., in particular from 10 to 40° C. The reaction specifically takes place at room temperature.

It can be carried out for example by thoroughly mixing a mixture of enantiomers of the trans or of the cis stereoisomers of the compounds of the formula (I) with the hydrolase, the acylating agent and, if appropriate, the solvent, e.g. by stirring or shaking. However, it is also possible to immobilize the hydrolase in a reactor, for example in a column, and to pass a mixture comprising the mixture of enantiomers and the acylating agent through the reactor. It is possible for this purpose to circulate the mixture through the reactor until the desired conversion is reached. During this, the carboxyl groups of the acylating agent are sequentially converted into amides of that enantiomer of the compound (I) which is enantioselectively acylated, while the other enantiomer remains substantially unchanged. The acylation will ordinarily be carried out until the conversion is at least 95%, preferably at least 98% and in particular at least 99%, based on the enantiomer of the compound (I) which is present in the mixture and which is enantioselectively acylated. The progress of the reaction, i.e. the sequential amide formation, can moreover be followed by usual methods such as gas chromatography or HPLC (high performance liquid chromatography).

The reaction mixture can be worked up in a conventional way, for example by removing the hydrolase if appropriate from the reaction mixture, e.g. by filtration or centrifugation, removing the solvent from the filtrate or centrifugate, and then subjecting the residue to a separation operation.

In a specific embodiment of the process of the invention, the mixture of enantiomers employed is the racemate of the amine (I); mixtures in which one of the enantiomers is enriched are, however, likewise suitable.

The enantioselective reaction of the mixture of enantiomers of the trans or cis isomers of the compound (I) results in a reaction product which comprises a substantially acylated enantiomer (i.e. amide) of the compound (I) and the substantially nonacylated opposite enantiomer. This mixture of amine and amide now present can easily be separated by conventional methods. Examples of suitable separation operations are extraction, distillation, crystallization or chromatography. The separation of the amine and amide preferably takes place by distillation. In an alternatively preferred separation process, the reaction mixture which is dissolved or suspended in an organic solvent is mixed with an acidic salt former to form and precipitate the ammonium salt of the nonacylated enantiomer. This can then be separated by filtration or centrifugation from the supernatant which comprises the amide of the opposite enantiomer of the compound (I). Suitable acidic salt formers are for example protic acids, in particular mineral acids such as hydrochloric acid, hydrobromic acid, sulfuric acid or nitric acid, but also organic acids such as trifluoroacetic acid, trifluoromethanesulfonic acid or para-toluenesulfonic acid, and their alkali metal salts, but with preference for acids, especially mineral acids and in particular hydrochloric acid or sulfuric acid. The acid is preferably employed in approximately equimolar amount in relation to the amount of nonacylated enantiomer, e.g. in an amount of from 0.9 to 1.5 mol, preferably 1 to 1.2 mol and in particular about 1 mol, based on 1 mol of the nonacylated enantiomer. In the case of polyprotic acids such as sulfuric acid, the molar ratios are of course based on the number of acidic protons present in the acid.

The substantially nonacylated enantiomer of the compound (I) which has been removed can if appropriate be subjected to a further purification using methods known to the skilled worker. The nonacylated enantiomer which is removed subsequent to the acylation is in particular (S,S)-2-benzyloxycyclohexylamine ((S,S)-I) [i.e. in step (b) of process B (S,S)-2-benzyloxycyclohexylamine ((S,S)-I) in particular is obtained].

If the resulting substantially nonacylated enantiomer of the compound (I) is, owing to the separation method, in the form of the ammonium salt, the amine is liberated from the ammonium salt using a suitable base. Examples of suitable bases are alkali metal hydroxides such as sodium hydroxide or potassium hydroxide, alkaline earth metal hydroxides such as calcium hydroxide or magnesium hydroxide, alkali metal carbonates or alkaline earth metal carbonates such as sodium carbonate, potassium carbonate or calcium carbonate. Alkali metal hydroxides such as sodium hydroxide or potassium hydroxide are preferably used. The neutralization preferably takes place in an aqueous medium. To facilitate isolation of the amine, the base is preferably employed in an amount such that the liberated compound is in neutral form. The free amine obtained can subsequently if desired be subjected to further purification steps.

The other enantiomer which has been enantioselectively acylated in the process of the invention can be obtained by the substantially acylated enantiomer of the compound (I) which has been obtained in the acylation, i.e. the amide of the compound (I), being hydrolyzed with elimination of the acyl function, resulting in the corresponding enantiomer of the compound (I). The product obtained is preferably (R,R)-2-benzyloxy-cyclohexylamine ((R,R)-I) [i.e. in step (c) of process B preferably (R,R)-2-benzyloxycyclohexylamine ((R,R)-I) is obtained].

The hydrolysis in this case usually takes place under reaction conditions as are known for the hydrolysis of amides. Reaction conditions are described for example in DE-A-19534208 or in Organikum, VEB Deutscher Verlag der Wissenschaften, Berlin 1988, 17th edition, p. 419 or in Jerry March, Advanced Organic Chemistry, 3rd edition, John Wiley and Sons, pp. 338 et seq., which are incorporated herein by reference. The hydrolysis to give the amine preferably takes place by reaction with a base. Examples of suitable bases are alkali metal hydroxides such as sodium hydroxide and potassium hydroxide, alkaline earth metal hydroxides such as calcium hydroxide, alkali metal and alkaline earth metal carbonates such as sodium, potassium and calcium carbonate, ammonia, amines such as dimethylamine, trimethylamine, diethylamine, triethylamine, diisopropylamine and diisopropylethylamine, or amino alcohols such as ethanolamine, diethanolamine and triethanotamine. The alkali metal hydroxides mentioned are particularly preferably used, if appropriate in combination with an amine or amino alcohol.

The hydrolysis can be carried out in water or in an organic solvent or in a mixture of water and organic solvent. Suitable organic solvents are alcohols, preferably having 1 to 3 carbon atoms, such as methanol, ethanol, propanol or isopropanol, glycols, in particular having 2 to 8 carbon atoms, such as ethylene glycol, di- and triethylene glycol, amines and amino alcohols, e.g. the aforementioned amines and amino alcohols, as well as the mixtures of the aforementioned solvents, and their mixtures with water. The hydrolysis preferably takes place at elevated temperature, e.g. at the boiling point of the solvent used.

The reaction product can be purified by usual processes, for example by distillation, sublimation, extraction or chromatography.

If the resulting hydrolyzed enantiomer of the compound (I) is in the form of the ammonium salt, for example because the acylated product has been hydrolyzed with acids, the amine is liberated from the ammonium salt using a suitable base as described above for the substantially nonacylated enantiomer. In this case too it is possible if appropriate to subject the free amine obtained subsequently to further purification steps known to the skilled worker.

It is also possible analogously on use of the cis stereoisomer to prepare the (R,S) and (S,R) enantiomers of the compound (I).

In a preferred embodiment of the process of the invention, the optically active compounds of the formulae ((R,R)-I) or ((S,S)-I) obtained in step (b) or (c) of process B are if desired to increase the chemical and/or optical purity converted into their ammonium salts by adding an acidic salt former (step (d)), isolated as such (step (e)) and subsequently liberated again as free base (step (f)). These steps are particularly appropriate when the optical or chemical purity of the enantiomers obtained in steps (b) and (c) is not yet satisfactory.

The salt formation advantageously takes place with precipitation of the acid addition salt of the compounds ((R,R-I) or ((S,S)-I). Concerning the procedure and in particular the selection of the acidic salt former, the statements made above about the separation by precipitation of the substantially nonacylated enantiomer (step (b)) apply.

For this purpose, the enantiomer to be purified is preferably introduced into an organic solvent, and the acidic salt former is added to the solution. It is, of course, also possible to add the enantiomer to the acidic salt former, if appropriate in the solvent, but the first variant is preferred. The solvent is preferably chosen so that the free base, i.e. the amine form of the enantiomer to be purified (R,R)-I) or ((S,S)-I), is soluble therein, but the ammonium salt thereof is essentially not, i.e. a maximum of 5%, preferably a maximum of 2% and in particular a maximum of 1%. Examples of suitable solvents are aliphatic and alicyclic hydrocarbons, preferably having 5 to 8 carbon atoms, such as pentane, cyclopentane, hexane, cyclohexane, heptane, octane or cyclooctane, halogenated aliphatic hydrocarbons, preferably having 1 or 2 carbon atoms, such as dichloromethane, chloroform, tetrachloromethane, dichloroethane or tetrachloroethane, aromatic hydrocarbons, such as benzene, toluene, the xylenes, chlorobenzene or dichlorobenzene, aliphatic acyclic and cyclic ethers, preferably having 4 to 8 carbon atoms, such as diethyl ether, methyl tert-butyl ether, ethyl tert-butyl ether, dipropyl ether, diusopropyl ether, dibutyl ether, tetrahydrofuran or dioxane, or mixtures of the aforementioned solvents. The aforementioned ethers and aromatic hydrocarbons are particularly preferably employed. Toluene is used specifically.

It is particularly preferred to add the acidic salt former in the form of an aqueous solution in the precipitation of the ammonium salts of the compound of the formula (I). Aqueous solutions of mineral acids such as hydrochloric acid, hydrobromic acid, sulfuric acid, nitric acid or phosphoric acid are preferably used. Hydrochloric acid is particularly suitable as acidic salt former. This is surprising because, despite the presence of water, a selective precipitation of the ammonium salt of the enantiomer to be purified takes place with negligible losses of yield.

In this specific embodiment of the process of the invention, it may be advantageous for the water added with the acidic salt former to be removed again, completely or at least in part, by a method known to the skilled worker. Suitable for this purpose, depending on the solvent, is for example azeotropic distillation.

It is advantageously possible to influence in a targeted manner the purity and yield of the salt of the compound of the formula (I) obtained from the precipitation, depending on the specifications to be met by the final product, via the water content of the reaction mixture.

The ammonium salts formed are isolated by generally customary processes for removing precipitated solids, e.g. by filtration, sedimentation, centrifugation, etc. with or without previous removal of the liquid phase present in the reaction mixture. The latter can be removed for example by decantation or distillation. The removed ammonium salt can if desired be subjected to further purification steps such as washing or digestion with a solvent in which the salt is insoluble.

The substantially enantiomerically pure compounds (I) can, after isolation of the precipitated product, be liberated again from their ammonium salts by neutralization with a base. The statements made about suitable bases for step (b) apply here correspondingly. The neutralization takes place in particular with an aqueous NaOH solution.

It is possible by this preferred embodiment to increase further the optical and chemical purity of the substantially enantiomerically pure compounds (I) (in particular (R,R)-I and (S,S)-I). The chemical and/or optical purity of optically active compounds of the formula (I) which are not, because their purity is too low, referred to as substantially enantiomerically pure compounds (I) in the context of the present invention can also be increased in this manner.

It is possible by this preferred embodiment to achieve chemical and optical purities of the 2-benzyloxycyclohexylamine enantiomers of respectively at least 99% and at least 99% ee, preferably at least 99.5% and at least 99.5% ee and in particular at least 99.9% and at least 99.9% ee.

The yields which can be achieved in the precipitation of the compounds (I) in the form of their ammonium salts by addition of an acidic salt former to increase the chemical and/or optical purity depend on the chemical and optical purity of the starting compounds and on the desired purities of the product obtained therefrom. The loss of yield is preferably less than 15% and in particular less than 10% based on the total amount of enantiomer to be isolated.

In a further preferred embodiment of the process of the invention, the trans stereoisomer of the compound of the formula (I) is obtained by:

(i) reacting cyclohexene oxide (epoxycyclohexane) with ammonia to result in the compound of the formula (II)

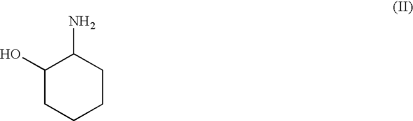

and

(ii) reacting the trans stereoisomer of the compound of the formula (II) with a suitable benzyl compound to give the trans stereoisomer of the compound of the formula (I)

or

(iii) reacting a mixture of cis- and transisomers of the compound of the formula (II) with a suitable benzyl compound to form a mixture of the corresponding cis- and trans-benzyl ethers (i.e. to form a mixture of the trans- and cisisomers of compound I) and isolating therefrom the trans-stereoisomer of the compound of the formula (I).

The preparation, referred to as step (I) in the context of the present invention, of aminocyclohexanol from cyclohexene oxide can be carried out for example by the method published by Schlichter and Frahm (Arch. Pharm. (Weinheim) 326, 429-436 (1993)). In this, cyclohexene oxide is added to an aqueous 35-40% strength ammonia solution and stirred overnight, and the solution is evaporated to dryness. In this case the reaction generally proceeds stereospecifically and results selectively in trans-2-aminocyclohexanol.

Conversion of the compound (II) into the corresponding benzyl ether takes place in step (ii) by reacting the trans-isomer of compound (II) with a suitable benzyl compound. Suitable benzyl compounds are for example benzyl halides, e.g. benzyl bromide or benzyl chloride. The reaction is usually carried out in the presence of a base. Suitable bases are the aforementioned alkali metal and alkaline earth metal hydroxides, and alkali metal and alkaline earth metal carbonates. The mixture of enantiomers obtained in this way can be isolated and purified if appropriate by methods known to the skilled worker. Methods of distillation and extraction are particularly suitable for this purpose.

The reaction sequence described above usually results in step (i) in substantially the trans stereoisomer of the compound (II) and in the subsequent step (ii) in substantially the trans stereoisomer of the compound (I), in particular with a diastereomeric excess of at least 99% de. The isolated mixture of enantiomers is specifically the racemate of the trans diastereomer of the compound (I). If, however, the reaction mixture in step (i) comprises a not inconsiderable amount of cis isomer, i.e. more than 1% based on the total weight of the cis/trans isomers, this is preferably removed from the trans isomer by suitable processes. The reaction mixture from step (i) is usually subjected to the separation process; i.e. the reaction mixture of step (i) is freed of the cis isomer before step (ii) is carried out; alternatively, however, it is also possible first to subject the mixture obtained in step (i) to step (iii), i.e. first reacting the cis/trans mixture with a suitable benzyl compound and then separating the reaction mixture of this benzylation reaction. The reaction mixture of the benzylation reaction comprises the trans and cis isomers of compound I; the trans isomer is isolated form this mixture. Regarding suitable and preferred benzyl compounds and reaction conditions for the benzylation, see the above observations. Suitable separation processes are known to the skilled worker and comprise for example methods of extraction and chromatography.

The trans stereoisomer of the compound (I) obtained in this way can be isolated and, if appropriate, further purified, for example by chromatography, extraction or by precipitation as ammonium salt.

The individual enantiomers of the compound (I) can be obtained with a very high optical and chemical purity and in high yields using the process of the invention. Specifically, (R,R)-2-benzyloxycyclohexylamine is obtained by process (B) of the invention with an enantiomeric excess (ee value) of preferably at least 98% ee, particularly preferably of at least 99% ee, more preferably at least 99.5% ee and in particular of at least 99.9% ee. The enantiomeric purity of the corresponding (S,S) enantiomer is preferably at least 97% ee, particularly preferably at least 98% ee, more preferably at least 99% ee, even more preferably at least 99.5% ee and in particular at least 99.9% ee. The chemical purity of the enantiomers is preferably at least 97%, particularly preferably at least 98%, more preferably at least 99%, even more preferably at least 99.5% and in particular at least 99.9%.

The enantiomeric excess of the amines ((R,R)-I) and ((S,S)-I) can be determined by conventional processes, for example by determining the optical rotation or by chromatography on a chiral phase, for example by HPLC or gas chromatography on chiral columns.

The present invention is illustrated by the following, non-restrictive examples.

### EXAMPLES

### 1.) Enantioselective Acylation and Racemate Resolution

Racemic trans-2-benzyloxycyclohexylamine (1537 g, 7.49 mol), consisting of equal parts of (R,R)-2-benzyloxycyclohexylamine and (S,S)-2-benzyloxycyclohexylamine, was mixed with isopropyl methoxy acetate (IPMA) (425 g, 3.97 mol, 0.53 equivalents) and cooled to 15° C., and then Novozyme 435® (30 g) was added. After removal of the cooling bath, the reaction solution was stirred overnight, the stirring speed being adjusted so that the enzyme just remained suspended. After 20 hours, the enzyme was filtered off, the residue on the filter was washed with toluene (0.5 l), and the filtrate was freed of volatile constituents in vacuo (15 mbar) at a maximum temperature of 50° C. The distillation residue was taken up in toluene (2.5 l) and, while cooling, adjusted to a pH of 1 by adding 10% strength sulfuric acid (about 1.8 l), taking care that the temperature did not exceed 20° C. The aqueous phase was separated off and extracted twice with toluene (200 ml each time). The combined organic extracts were washed with 10% strength sulfuric acid (100 ml) and water (200 ml). The solvent was then removed under reduced pressure, and the distillation residue was freed of low-boiling constituents at 0.5 mbar and a bath temperature of 180° C. (R,R)-N-(2-benzyloxycyclohexyl)methoxyacetamide (920 g, 89%) was obtained as residue as highly viscous oil. Recrystallization from hot cyclohexane and n-pentane afforded the product as crystalline solid (melting point 45-48° C.). The ee was >99.9%. The (S,S) enantiomer was present substantially in the form of its salt in the combined aqueous phases of the extraction.

¹H-NMR (400 MHz, CDCl₃) of (R,R)-N-(2-benzyloxycyclohexyl)methoxy-acetamide: [symbol] =1.22 (m, 2H); 1.38 (m, 2H), 1.58 -1.68 (m, 1H), 1.80 (m, 2H), 2.15 (m, 2H), 3.25 (m, 1H), 3.40 (s, 3H); 3.84 and 3.92 (J_{AB}=20 Hz, 2H), 3.05 (m, 1H), 4.48 and 4.68 (J_{AB}=16 Hz, 2H), 6.55 (s, broad, 1H), 7.22-7.40 (m, 5H).

### 2.) Isolation of the Enantiomers

2.1) Isolation of (S,S)-2-benzyloxycyclohexylamine

The combined aqueous phases obtained in 1 were mixed with toluene (2 l). The pH was adjusted to about 13 with aqueous NaOH solution (50% strength), keeping the mixture at a temperature of less than 30° C. by cooling, After filtration through glass wool, the aqueous phase was separated off and extracted twice with toluene (200 ml each time). The combined organic phases were washed with water (200 ml). The aqueous phase was then discarded and the solvent of the organic phases was removed in a rotary evaporator. A brown oil (950 g) was obtained as residue which comprised, besides solvent residues, as main constituent 87% (S,S)-2-benzyloxycyclohexylamine. Distillation under reduced pressure (0.5 mbar/100° C.) afforded (S,S)-2-benzyloxycyclohexylamine (653 g, 85%) with a chemical purity of 97% and an ee of 97.3%.

¹H-NMR (400 MHz, CDCl₃): δ=1.08-1.38 (m, 4H), 1.58-1.82 (m, 4H), 1.90 (m, 1H), 2.25 (m, 1H), 2.65 (m, 1H), 3.05 (m, 1H), 4.45 and 4.70 (J_{AB}=16 Hz, 2H), 7.22-7.40 (m, 5H).

2.2) Preparation of (R,R)-2-benzyloxycyclohexylamine

(R,R)-N-(2-benzyloxycyclohexyl)methoxyacetamide (1687 g, 6.08 mol) was diluted with triethanolamine (300 g) and, while stirring, aqueous sodium hydroxide solution (50% strength, 780 g, 9.75 mol) was added. The mixture was heated to an internal temperature of 120° C. and vigorously stirred. After 7 hours, the reaction mixture was diluted with water (500 ml). After cooling to room temperature, toluene (2 l) was added, and the phases were separated from one another. The aqueous phase was again extracted with toluene (200 ml), the organic phases were combined, and the combined organic phases were washed with water (200 ml). After removal of the aqueous phase, the solvent was removed in a rotary evaporator, and the residue was fractionally distilled under reduced pressure (boiling point 100° C. under 0.4 to 0.5 mbar). (R,R)-2-Benzyl-oxycyclohexylamine was obtained as a clear colorless liquid in a yield of 1125 g (90%). The ee was >99.9%.

Rotation [α]_{D}=−96.8° (c=2 in methanol)

¹H-NMR spectrum identical to that of the (S,S) enantiomer

3.) Increase in the Chemical and/or Optical Purity

These examples which follow serve to illustrate the great efficiency of the precipitation according to the invention.

3.1) Precipitation of (S,S)-2-benzyloxycyclohexylamine Hydrochloride

(S,S)-2-Benzyloxycyclohexylamine with a chemical and optical purity of respectively 98.5% and 89.6% ee (adjusted by admixture of the (R,R) enantiomer) was introduced into toluene (90 ml) and, while stirring, concentrated hydrochloric acid was added. After the addition was complete, the mixture was heated to reflux and the distillate was returned to the reaction vessel via a water trap filled with toluene, After removal of water was complete, the water trap was replaced by a reflux condenser, and isopropanol (10 ml) was added. After cooling to room temperature, the precipitated solid was isolated by filtration, washed with cold toluene (10 ml) and dried under reduced pressure. The hydrochloride of (S,S)-2-benzyloxycyclohexylamine was obtained as a colorless solid (20.1 g, 83%) with a melting point of 182-184° C. A sample of the free 2-benzyloxycyclohexylamine liberated by aqueous sodium hydroxide solution had a chemical and optical purity of respectively >99.9% and >99.9% ee.

3.2) Precipitation of (S,S)-2-benzyloxycyclohexylamine Hydrochloride

Application of the method of example 3.1) using (S,S)-2-benzyloxycyclohexylamine with an optical purity of 85% ee (adjusted by admixture of the (R,R) enantiomer) resulted in a hydrochloride in a yield of 82.5% with an optical purity of 95% ee.

Repetition of the precipitation led to a product with >99% ee.

Rotation of the ammonium salt: [α]_{D}+78.8° (c=2 in methanol)

¹H-NMR (400 MHz, CDCl₃) of the hydrochloride: δ=1.05-1.38 (m=3H), 1.70 (m, 3H), 2.10 (m, 1H), 2.35 (m, 1H), 3.05 (m, 1H), 3.55 (m, 1H) 4.50 and 4.70 (J_{AB}=16 Hz, 2H), 7.25-7.50 (m, 5H), 8.50 (s, broad, 3H).

3.3) Precipitation of (R,R)-2-benzyloxycyclohexylamine hydrochloride

Starting from (R,R)-2-benzyloxycyclohexylamine with a chemical purity of 96% and an optical purity of >99.9% ee, the hydrochloride (324.3 g, 85%) was obtained as a colorless solid. A sample of the free amine liberated by aqueous sodium hydroxide solution had a chemical and optical purity of respectively >99.9% and >99.9% ee.

1-15. (canceled)

16. A process for enantioselectively acylating trans- or cis-2-benzyloxycyclohexylamine (I) comprising reacting a mixture of enantiomers of the trans stereoisomer or of the cis stereoisomer of the compound of formula (I) 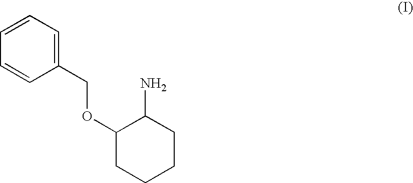
with an acylating agent in the presence of a hydrolase to form a mixture wherein one enantiomer of the trans or of the cis stereoisomer is present substantially in the acylated form and the other enantiomer of the trans or of the cis stereoisomer is present substantially in the nonacylated form.

17. The process of claim 16, wherein a mixture of enantiomers of the trans stereoisomer of the compound of formula (I) is used and a mixture of substantially acylated (R,R) enantiomer and substantially nonacylated (S,S) enantiomer is obtained.

18. A process for preparing optically active compounds of the formulae ((R,R)-I) and/or ((S,S)-I) 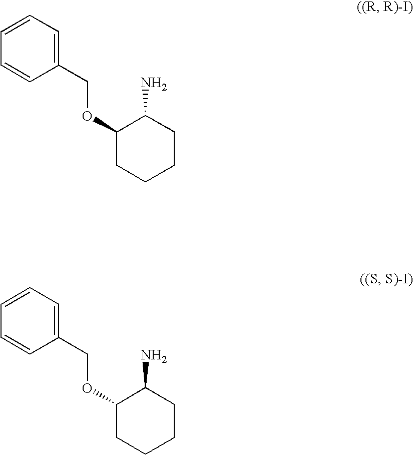
comprising:
(a) reacting a mixture of enantiomers of the trans stereoisomer of the compound of formula (I): 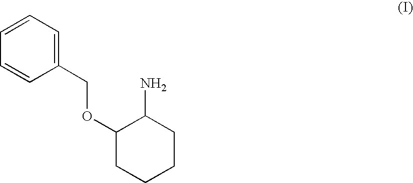
with an acylating agent in the presence of a hydrolase to form a mixture wherein one enantiomer of the trans stereoisomer of the compound of formula (I) is present substantially in the acylated form and the other enantiomer of the trans stereoisomer of the compound of formula (I) is present substantially in the nonacylated form;
(b) removing the nonacylated enantiomer of the trans stereoisomer of the compound of formula (I) from the mixture obtained in (a);
(c) hydrolyzing the acylated enantiomer of the trans stereoisomer of the compound of formula (I) obtained in (b) to the corresponding nonacylated enantiomer of the compound of formula (I).

19. The process of claim 18, wherein the substantially nonacylated enantiomer obtained in (a) is the (S,S) enantiomer of the compound of formula (I) and the substantially acylated enantiomer is the (R,R) enantiomer of the compound of formula (I).

20. The process of claim 16, wherein said acylating agent is an ester whose acid component has an oxygen-, nitrogen-, fluorine-, or sulfur-containing group in the α, β, or γ position relative to the carbonyl carbon atom.

21. The process of claim 16, wherein said acylating agent is employed in an amount of from 1.0 to 1.5 equivalents based on the molar amount of the enantiomer to be acylated.

22. The process of claim 16, wherein said hydrolase is selected from the group consisting of lipases from bacteria of the genus Burkholderia, lipases from bacteria of the genus Pseudomonas, and lipases from yeasts of the genus Candida.

23. The process of claim 22, wherein said lipase is Lipase B from Candida antarctica.

24. The process of claim 16, wherein said reaction with said acylating agent is carried out in the presence of the hydrolase without addition of solvents.

25. The process of claim 16, where said reaction with said acylating agent is carried out in the presence of the hydrolase in a nonaqueous reaction medium.

26. The process of claim 18, further comprising the following steps:
(d) converting the optically active compounds of formulae ((R,R)-I) or ((S,S)-I) obtained in (b) or (c) into their ammonium salts by adding an acidic salt former;
(e) isolating said ammonium salts; and
(f) liberating the free bases of the optically active compounds of formulae ((R,R)-I) or ((S,S)-I) from the ammonium salts.

27. The process of claim 26, wherein said acidic salt former is added in the form of an aqueous solution to form a reaction solution.

28. The process of claim 27, wherein water is at least partly removed from said reaction solution after addition of said aqueous solution.

29. The process of claim 26, where said acidic salt former is selected from the group consisting of protic acids and their alkali metal salts.

30. The process of claim 16, wherein the trans stereoisomer of the compound of formula (I) is obtained by:
(i) reacting cyclohexene oxide with ammonia to form the compound of formula (II) 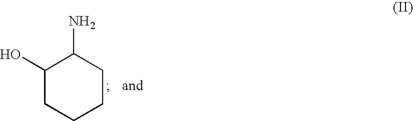
(ii) reacting the trans stereoisomer of the compound of formula (II) with a benzyl compound to form the trans stereoisomer of the compound of formula (I): 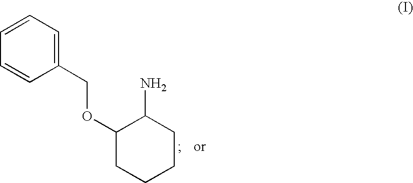
(iii) reacting a mixture of cis and trans isomers of the compound of formula (II) with a benzyl compound to form a mixture of the corresponding cis- and trans-benzyl ethers and isolating therefrom the trans-stereoisomer of the compound of formula (I).
